(19) United States
(12) Patent Application Publication
(10) Pub. No.: US 2024/0142471 A1
(43) Pub. Date: May 2, 2024

(54) TEST FOR MILD COGNITIVE IMPAIRMENT

(71) Applicant: OSAKA UNIVERSITY, Suita-shi, Osaka (JP)

(72) Inventors: Masuo KONDOH, Suita-shi, Osaka (JP); Keisuke TACHIBANA, Suita-shi, Osaka (JP); Ryuichi HIRAYAMA, Suita-shi, Osaka (JP)

(21) Appl. No.: 18/278,931

(22) PCT Filed: Feb. 9, 2022
(86) PCT No.: PCT/JP2022/005075

§ 371 Date: Aug. 25, 2023

(30) Foreign Application Priority Data
Feb. 26, 2021 (JP) 2021-031160

(51) Int. Cl. G01N 33/68

(52) U.S. Cl. CPC G01N 33/6896; G01N 2333/705; G01N 2800/2821; G01N 2800/2835; G01N 2800/285; G01N 2800/301; G01N 2800/304

(57) ABSTRACT

Provided is a means for detecting a subject with mild cognitive impairment or assisting in the detection thereof.

A biomarker for testing for mild cognitive impairment, comprising Claudin-5.

### TECHNICAL FIELD

Techniques relating to testing or diagnosing for mild cognitive impairment are disclosed.

### BACKGROUND ART

Japan is facing a rapid population decline and rapid aging of the population resulting from the decline in the birthrate. It is estimated that the population aging rate will continue to rise even after 2042, at which point the number of elderly people will reach a peak, and that one out of every 2.6 people in Japan will be 65 years old or more in 2065. While the average life expectancy is 81 years for men and 87 years for women, the healthy life expectancy, during which a person can live independently without the need for nursing care, is 72 years for men and 75 years for women. There is a 10-year gap between the average life expectancy and the healthy life expectancy. Thus, in order to maintain a sustainable society in the future super-aging society, the most important issue is to extend the healthy life expectancy so that people can live healthy lives without receiving nursing care.

Dementia is one of the reasons for starting nursing care. One out of every seven elderly people is said to have dementia, and it is estimated that the number will exceed 7 million in 2025. The number of dementia patients in the world exceeds 50 million, and it is estimated that the number will reach 150 million in 2050. At the 2013 London G8 Summit, dementia was taken up as a major topic of discussion, and measures against dementia have been advanced on a global scale. Drugs that suppress cognitive decline have already been put on the market, and if treatment intervention can be provided at an early stage, it is possible to extend healthy life expectancy by suppressing the progression of dementia. However, highly reproducible objective biomarkers that contribute to early treatment intervention have not yet been put into practical use, which is a major hindrance to therapeutic development. Thus, drug contribution and treatment satisfaction for dementia remains at the lowest level among all diseases, and the number of patients continues to increase.

Dementia develops after more than 30 years through the conditions from the preclinical stage to mild cognitive impairment (MCI), which are the presymptomatic stage, and current dementia treatment is targeted at dementia patients and uses drugs that activate nerve cells in the cerebral cortex and hippocampus, which control cognitive function. However, in dementia, neurodegeneration has progressed, and there are not enough nerve cells remaining; thus, once the degeneration of nerve cells progresses, the therapeutic effect of drugs for dementia cannot be expected. On the other hand, neurodegeneration has not progressed in a presymptomatic state, and it is believed that if a therapeutic drug can be administered to a person in a presymptomatic state, it would be possible to suppress cognitive decline and delay or prevent the onset of dementia. In other words, the most important strategy for measures against dementia is to detect people at risk of developing dementia in a presymptomatic stage and provide early prevention and treatment intervention. However, no objective biomarkers for detecting people at risk of developing dementia in a presymptomatic stage have been put into practical use. Moreover, cognitive decline is tested by a questionnaire; thus, sufficient preventive and therapeutic effects cannot be expected in patients who diagnosed with dementia, in whom neurodegeneration has progressed. Therefore, a means for detecting people in a presymptomatic state (preclinical stage and MCI) is required for the prevention and treatment of dementia.

In Alzheimer's disease (AD), which accounts for about 70% of dementia, it is known that many senile plaques, which are aggregates of amyloid and tau proteins, are formed in the brain and that albumin enters the brain by breakdown of the blood-brain barrier (BBB) (Non-patent Literature (NPL) 1). AD progresses in the following order: accumulation of amyloid and tau proteins in the brain, brain atrophy, and cognitive decline. Since the deposition of amyloid and tau proteins in the brain occurs before cognitive decline, the development of dementia biomarkers using senile plaques or the like as an indicator has been advanced. However, although a technique for analyzing amyloid and tau proteins in cerebrospinal fluid has been established, cerebrospinal fluid collection is highly invasive and basically requires hospitalization (rest for 2 to 3 hours after collection). In addition to side effects such as headache, nausea, and vomiting, there are no small number of cases of blood contamination of specimens and difficulty in lumbar puncture due to lumbar spondylosis in elderly people. This technique has problems in terms of convenience and versatility. It is thus difficult to put this technique to practical use as a screening technique for people in a presymptomatic state (MCI).

### Citation List

### Patent Literature

PTL 1: W02018/207638

PTL 2: W02018/105560

### Non-patent Literature

NPL 1: Zenaro, E. et al., The blood-brain barrier in Alzheimer's disease. Neurobiol Dis 2017, 107, 41-56, doi:10.1016/j.nbd.2016.07.007.

NPL 2: Hashimoto et al., Claudin-5-Binders Enhance Permeation of Solutes across the Blood-Brain Barrier in a Mammalian Model. J Pharmacol Exp Ther 2017, 363, 275-283

NPL 3: Hashimoto et al., Engineered membrane protein antigens successfully induce antibodies against extracellular regions of claudin-5. Sci Rep 2018, 8, 8383

### SUMMARY OF INVENTION

### Technical Problem

Under such circumstances, an object of the present invention is to provide a means for detecting a subject with mild cognitive impairment or assisting in the detection thereof.

### Solution to Problem

Item 1.

A biomarker for testing for mild cognitive impairment, comprising Claudin-5.

Item 2.

The biomarker according to Item 1, wherein the testing for mild cognitive impairment comprises distinguishing mild cognitive impairment from a group of diseases consisting of Parkinson's disease, multiple sclerosis, Alzheimer's disease, obsessive-compulsive disorder, and bipolar disorder.

Item 3.

A method for testing for mild cognitive impairment in a subject using Claudin-5 in a body fluid collected from the subject as an indicator.

Item 4.

The method according to Item 3, wherein the testing comprises distinguishing mild cognitive impairment from a group of diseases consisting of Parkinson's disease, multiple sclerosis, Alzheimer's disease, obsessive-compulsive disorder, and bipolar disorder.

Item 5.

The method according to Item 3 or 4, comprising measuring the amount of Claudin-5 in the body fluid collected from the subject.

Item 6.

The method according to Item 5, wherein the amount of Claudin-5 in the body fluid is measured using an anti-Claudin-5 monoclonal antibody.

Item 7.

The method according to any one of Items 3 to 6, comprising comparing the amount of Claudin-5 in the body fluid of the subject with the amount of Claudin-5 in a body fluid of a healthy subject.

Item 8.

The method according to any one of Items 3 to 7, wherein the subject is a subject suspected of having cognitive impairment.

Item 9.

The method according to any one of Items 3 to 8, further characterized by being combined with one or more diagnoses selected from the group consisting of a medical interview, a brain image analysis, a phosphorylated tau test, an amyloid β test, an electrophysiology study, a cerebrospinal fluid examination, a urinalysis, a blood test, a chest X-ray examination, a cerebral blood flow SPECT test, a brain glucose metabolism PET test, and a genetic test, with the proviso that a medical practice is excluded.

Item 10.

A reagent for use in performing the method according to any one of Items 3 to 9, comprising an anti-Claudin-5 antibody.

Item 11.

A method for obtaining information about cognitive impairment in a subject using a substance in a sample as an indicator, the method comprising:
mixing an anti-Claudin-5 antibody with a body fluid collected from a subject to prepare a sample; and detecting a substance to which the anti-Claudin-5 antibody has bound in the sample.

Item 12.

A reagent for use in performing the method according to Item 11, comprising an anti-Claudin-5 antibody.

### Advantageous Effects of Invention

The present invention makes it possible to detect (or assist in detecting) a subject with mild cognitive impairment.

### BRIEF DESCRIPTION OF DRAWINGS

FIG. 1 shows the results of detection of Claudin-5 micelles using monoclonal antibodies that recognize the extracellular region of Claudin-5.

FIG. 2 shows the results of examining the Claudin-5 detection specificity of an enzyme immunoassay using monoclonal antibodies that recognize the extracellular region of Claudin-5.

FIG. 3 shows the results of confirming that Claudin-5 in a human biological sample can be quantitatively measured by an enzyme immunoassay using a monoclonal antibody that recognizes the extracellular region of Claudin-5.

### DESCRIPTION OF EMBODIMENTS

Claudin-5 is a protein that is an expression product of Claudin-5 (also referred to as CLDN-5, Cldn-5, CLDN5, Cldn5, or the like) gene and is expressed in organisms. The amino acid sequences of CLDN-5 proteins derived from various organism species are known.

As shown in the Examples described later, the amount of Claudin-5 in the blood of subjects with mild cognitive impairment is notably higher than that in the blood of healthy subjects or subjects with other neurodegenerative diseases (e.g., Alzheimer's disease, multiple sclerosis, and Parkinson's disease) or mental disorders (obsessive-compulsive disorder and bipolar disorder), and it is specifically detected in subjects with mild cognitive impairment. Thus, Claudin-5 in a body fluid can be used as a biomarker for the detection of subjects with mild cognitive impairment. Moreover, the use of the marker makes it possible to identify (or diagnose or determine) that a subject has mild cognitive impairment, rather than the neurodegenerative diseases or mental disorders described above.

The type of body fluid is not limited, and the body fluid is preferably blood or cerebrospinal fluid. The type of blood is not limited, and the blood is preferably peripheral blood. The site from which peripheral blood is collected is not limited, and examples include an arm, a leg, and a neck. In one embodiment, the blood is preferably blood drawn from the forearm. The blood may be whole blood, plasma, or serum, preferably plasma or serum, and more preferably serum.

The type of subject is not particularly limited, examples include animals including humans and mammals other than humans (e.g., dogs, cats, monkeys, chimpanzees, gorillas, horses, cows, and sheep). The subject is preferably a human.

In one embodiment, the subject is preferably a subject suspected of having cognitive impairment. The subject suspected of having cognitive impairment can be, for example, a subject who is subjectively or objectively suspected of showing a decline from a previous level of performance in one or more cognitive domains (complex attention, executive function, learning and memory, language, perceptual-motor, and social cognition). In one embodiment, the subject is preferably a certain age or more (e.g., 30 years old or more, 35 years old or more, 40 years old or more, 45 years old or more, 50 years old or more, 55 years old or more, 60 years old or more, 65 years old or more, 70 years old or more, or 75 years old or more).

Mild cognitive impairment is a condition that is neither dementia nor cognitively normal. For example, mild cognitive impairment has the following characteristics: (1) subjective or objective complaint of cognitive decline; (2) cognitive decline that does not meet the diagnostic criteria for dementia; (3) normal basic daily living function and independent living. According to the diagnostic criteria of the U.S. National Institute of Aging-Alzheimer's Association workgroup (NIA-AA), mild cognitive impairment is as follows: (A) cognitive decline from a previous level, which can be noted by self- or informant report, and/or a clinician; (B) impairment in two or more of the following cognitive domains: memory, executive function, attention, language, and visuospatial cognition; (C) independence in activities of daily living; they may take longer, be more inefficient, or be more error-prone, than in the past; (D) no dementia. Mild cognitive impairment is classified into amnestic mild cognitive impairment (amnestic MCI) and non-amnestic mild cognitive impairment (non-amnestic MCI) based on the presence or absence of memory impairment, and amnestic MCI is known to be likely to progress to dementia due to Alzheimer's disease (AD) (AD dementia).

As shown in the Examples described later, Claudin-5 is not detected in the blood of subjects with Parkinson's disease, multiple sclerosis, Alzheimer's disease, obsessive-compulsive disorder, or bipolar disorder, but is detected in the blood of subjects with mild cognitive impairment. Thus, it is possible to identify (or diagnose or determine) (or assist in identifying, or diagnosing or determining) that a subject has mild cognitive impairment, rather than Parkinson's disease, multiple sclerosis, Alzheimer's disease, obsessive-compulsive disorder, or bipolar disorder, using Claudin-5 in blood as an indicator.

In one embodiment, the testing for mild cognitive impairment means assisting in identifying (or diagnosing or determining) whether a subject has mild cognitive impairment. In one embodiment, the testing for mild cognitive impairment means assisting in identifying (or diagnosing or determining) whether a subject has mild cognitive impairment, based on Claudin-5 in the blood of the subject measured.

In one embodiment, provided is a method for testing for mild cognitive impairment in a subject using Claudin-5 in the blood of the subject as an indicator. This method can be used as a method for assisting in identifying (or diagnosing or determining) whether a subject has mild cognitive impairment. The testing method may include collecting a body fluid from a subject and/or measuring the amount of Claudin-5 in the body fluid collected from the subject. The method for collecting a body fluid from a subject and the method for measuring the amount of Claudin-5 in the body fluid may be any methods.

The method for measuring Claudin-5 in blood is not particularly limited, and for example, a measurement method using an immunological technique, high-performance liquid chromatography, liquid chromatography-tandem mass spectrometry, or the like can be used. In one embodiment, Claudin-5 in blood is preferably measured by an immunoassay using an anti-Claudin-5 antibody. The anti-Claudin-5 antibody used to measure Claudin-5 in blood is preferably a monoclonal antibody, preferably a monoclonal antibody that recognizes the extracellular region of Claudin-5. Examples of such monoclonal antibodies include monoclonal antibodies disclosed in PTL 1, PTL 2, NPL 2, NPL 3, and the like. The immunoassay may be any method.

The immunoassay is not particularly limited, and examples include an enzyme immunoassay, chemiluminescence immunoassay, fluorescence immunoassay, radioimmunoassay, immunoturbidimetry, immunoprecipitation, Western blotting, affinity chromatography, and the like. Among these, ELISA, which is an enzyme immunoassay, in particular, sandwich ELISA, is preferable.

The method for testing for mild cognitive impairment may comprise comparing the amount of Claudin-5 in the blood of the subject with the amount of Claudin-5 in the blood of a healthy subject. The healthy subject means an animal (preferably a human) with no past medical history of cognitive impairment that is of the same species as the subject that has cognitive impairment without a doubt objectively and subjectively. The method for testing for mild cognitive impairment can suggest that a subject is highly likely to have mild cognitive impairment when the amount of Claudin-5 in the blood of the subject measured is significantly higher than the amount of Claudin-5 in the blood of the healthy subject.

The method for testing for mild cognitive impairment can suggest whether a subject is highly likely to have mild cognitive impairment based on a predetermined cutoff value for the amount of Claudin-5 in blood, or can assist in identifying (or diagnosing or determining) whether a subject has mild cognitive impairment. The cutoff value can be determined, for example, based on the measured amount of Claudin-5 in the blood of a subject with mild cognitive impairment and the measured amount of Claudin-5 in the blood of a subject to be compared (which can include dementia patients) that does not have mild cognitive impairment. The cutoff value can be set by generating an ROC curve.

In one embodiment, the method for testing for mild cognitive impairment may be a method for assisting in identifying (or diagnosing or determining) that a subject is highly likely to have mild cognitive impairment or has mild cognitive impairment when the concentration of Claudin-5 in the blood of the subject is 0.001 ng/ml or more. The concentration of Claudin-5 in the blood may be 0.005 ng/ml or more, 0.01 ng/ml or more, 0.05 ng/ml or more, or 0.1 ng/ml or more.

The method for testing for mild cognitive impairment can be combined with other tests for mild cognitive impairment. This increases the accuracy of identification (or diagnosis or determination). Examples of the other tests include a medical interview, a brain image analysis, a phosphorylated tau test, an amyloid β test, an electrophysiology study, a cerebrospinal fluid examination, a urinalysis, a blood test, a chest X-ray examination, a cerebral blood flow SPECT test, a brain glucose metabolism PET test, a genetic test, and the like. One or more of these tests can be combined with the method for testing for mild cognitive impairment described above.

The medical interview includes cognitive function tests such as HDS-R (Hasegawa's Dementia Scale-Revised), Mini-Cog, MoCA (Montreal Cognitive Assessment), DASC-21 (Dementia Assessment Sheet for Community-based Integrated Care System-21 items), MMSE (Mini-Mental State Examination), and ABC-DS (ABC dementia scale).

In one embodiment, provided is a reagent for assisting in identifying (or diagnosing or determining) whether a subject has mild cognitive impairment in the method described above. The reagent can comprise a reagent for measuring Claudin-5 in blood (e.g., an anti-Claudin-5 antibody). The reagent can also comprise a buffer or any other component (e.g., a biotin-conjugated antibody, a streptavidin-conjugated particles, an enzyme-labeled antibody, an enzyme substrate, and a chemiluminescent substrate).

In one embodiment, provided is a method for obtaining information about cognitive impairment in a subject using a substance in a sample as an indicator, comprising mixing an anti-Claudin-5 antibody with a body fluid collected from a subject to prepare a sample, and detecting a substance to which the anti-Claudin-5 antibody has bound in the sample. In this method, the subject, the body fluid, and the anti-Claudin-5 antibody may be the same as those described above. The substance to which the anti-Claudin-5 antibody has bound can be a complex of the anti-Claudin-5 antibody and a substance to which it binds, and the substance to which the anti-Claudin antibody binds includes, for example, Claudin-5, fragments thereof, and exosomes containing them.

The information obtained by the method described above is not particularly limited, and includes, for example, information on whether Claudin-5 is detected in the body fluid or sample, information on the amount of the complex of the anti-Claudin-5 antibody and a substance to which it binds, information on the amount of Claudin-5 (including a fragment thereof) contained in the complex, and the like. Based on the information, it is possible to identify (or diagnose or determine) (or assist in identifying, or diagnosing or determining) cognitive impairment in the subject.

The anti-Claudin-5 antibody used in the above method may be an anti-Claudin-5 antibody alone or may be in the form of a reagent containing an anti-Claudin-5 antibody in combination with a buffer or any other component (e.g., a biotin-conjugated antibody, streptavidin-conjugated particles, an enzyme-labeled antibody, an enzyme substrate, and a chemiluminescent substrate), as necessary.

### Examples

The following Examples describe the present invention in more detail. However, the invention is not limited to the Examples.

### 1. Establishment of Immunoassay Using Anti-Claudin-5 Antibody

A quantification system using sandwich ELISA, which is an enzyme immunoassay, was established to detect Claudin-5 in blood, in consideration of versatility, quantitativeness, etc. As a model for Claudin-5 in blood, independently developed Claudin-5 micelles in which Claudin-5 is expressed on the membrane of liposomes using an modified wheat germ cell-free synthesis system was used (NPL 3). In addition, an antibody that recognizes the extracellular region of Claudin-5, which had been independently produced (PTL 1 and PTL 2), was used as an antibody for detecting Claudin-5. 100 μL of a solution prepared so as to contain 5 μg/mL of a capture antibody against Claudin-5 (the R9 antibody of PTL 1, the 2B12 antibody of PTL 2) was added to each well of a 96-well plate for ELISA (Nunc MaxiSorp™ flat-bottom (Thermo Fisher, 442404)), and a reaction was allowed to proceed at 25° C. for 1 hour to immobilize the capture antibody. After the reaction solution was removed, 300 μL of a blocking agent (Blocking One (Nacalai, 03953-95)) was added, and a reaction was allowed to proceed at 4° C. overnight. After the blocking agent was removed, the plate was washed three times with 300 μL of washing solution 1 (TBS-0.05% Tween20-1 mM DTT). After washing, 100 μL of reaction solution 1 (0.5% BSA-TBS-1 mM DTT) prepared so as to contain 10 ng/mL of Claudin-5 micelles or 100 ng/mL of any of Claudin-1 to Claudin-6 micelles was added, and a reaction was allowed to proceed at 25° C. for 1.5 hours. After the reaction solution was removed, the plate was washed three times with 200 μL of washing solution 1 and twice with 200 μL of washing solution 2 (TBS-1 mM

DTT). Subsequently, 50 μL of reaction solution 1 prepared so as to contain 0.5 μg/mL of a detection antibody against Claudin-5 (ab53765, EPR7583 (Abcam), SAB4502981 (Sigma-Aldrich)) was added, and a reaction was allowed to proceed at 25° C. for 1 hour. After the reaction solution was removed, the plate was washed five times with 200 pL of washing solution 2. Further, 50 pL of reaction solution 1 prepared so as to contain 50 ng/mL of a biotin-labeled secondary antibody (Biotin-SP-AffiniPure Donkey Anti-Rabbit IgG(H+L) (Jackson, 711-065-152)) was added, and a reaction was allowed to proceed at 25° C. for 1 hour. After the reaction solution was removed, the plate was washed five times with 200 pL of washing solution 2. After washing, 50 μL of reaction solution 1 prepared so as to contain 50 ng/mL of HRP-labeled streptavidin (Pierce High Sensitivity Streptavidin-HRP (Thermo Fisher, 21130)) was added, and a reaction was allowed to proceed at 25° C. for 30 minutes. After the reaction solution was removed, the plate was washed five times with 200 pL of washing solution 3 (TBS). Finally, 100 pL of a substrate (ABTS peroxidase substrate system (KPL, 50-62-00)) was added, and a reaction was allowed to proceed at 25° C. for 10 to 60 minutes. Thereafter, the absorbance at a wavelength of 405 nm was measured using a TriStar LB 941 plate reader (Berthold Technologies).

The R9 antibody used above comprises a heavy-chain CDR1 comprising the amino acid sequence of SEQ ID NO: 1, a heavy-chain

CDR2 comprising the amino acid sequence of SEQ ID NO: 2, a heavy-chain CDR3 comprising the amino acid sequence of SEQ ID NO: 3, a light-chain CDR1 comprising the amino acid sequence of SEQ ID NO: 4, a light-chain CDR2 comprising the amino acid sequence of SEQ ID NO: 5, and a light-chain CDR3 comprising the amino acid sequence of SEQ ID NO: 6. The 2B12 antibody used above comprises a heavy-chain CDR1 comprising the amino acid sequence of SEQ ID NO: 7, a heavy-chain CDR2 comprising the amino acid sequence of SEQ ID NO: 8, a heavy-chain CDR3 comprising the amino acid sequence of SEQ ID NO: 9, a light-chain CDR1 comprising the amino acid sequence of SEQ ID NO: 10, a light-chain CDR2 comprising the amino acid sequence of SEQ ID NO: 11, and a light-chain CDR3 comprising the amino acid sequence of SEQ ID NO: 12.

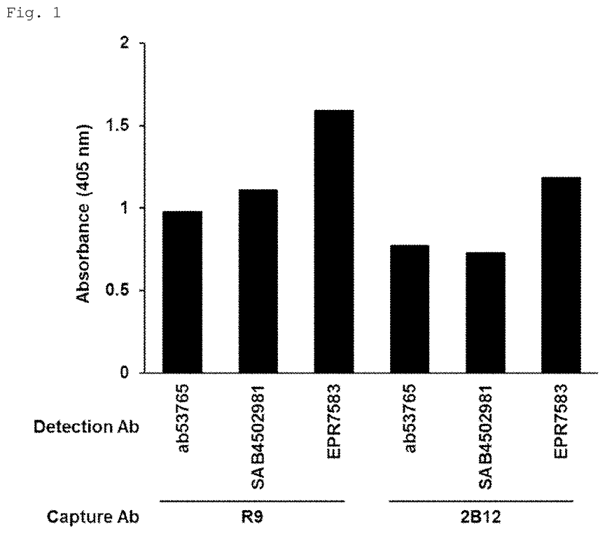

The results confirmed that all combinations of either R9 or 2B12, both of which are monoclonal antibodies that recognize the extracellular region of Claudin-5, as a capture antibody, with any of polyclonal antibody SAB4502981, monoclonal antibody EPR7583, and polyclonal antibody ab53765 (epitope unknown), all of which recognize the intracellular region, as a detection antibody can detect 10 ng/mL of Claudin-5 micelles (FIG. 1). In the following, monoclonal antibodies (R9 or 2B12 as a capture antibody and EPR7583 as a detection antibody), which are considered to have less lot-to-lot effect, were used.

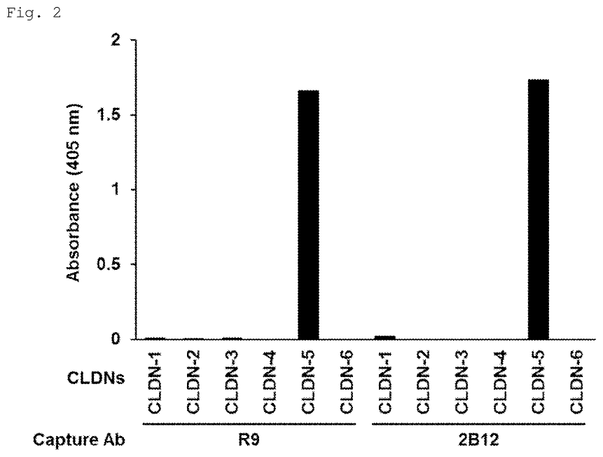

To evaluate the specificity of the ELISA method for Claudin-5, the ELISA method was performed using reaction solutions containing any of Claudin-1 to Claudin-6 as samples, and a signal was observed only when Claudin-5 micelles were used, with the use of either of the capture antibodies (FIG. 2). That is, it was confirmed that the immunoassay using an antigen-antibody reaction established in this study can specifically detect Claudin-5.

### 2. Analysis of Claudin-5 in Clinical Specimen Using Immunoassay

Next, the amounts of Claudin-5 in sera derived from patients with various central nervous system diseases were measured using commercially available clinical specimens. As samples for calibration curve, samples obtained by adding Claudin-5 micelles to pooled serum derived from healthy human subjects to a final concentration of 0.03125 to 8 ng/mL were used. 100 μL of a solution prepared so as to contain 5 μg/mL of a capture antibody (R9) against Claudin-5 was added to each well of a 96-well plate for ELISA (Nunc MaxiSorpTM flat-bottom (Thermo Fisher, 439454)), and a reaction was allowed to proceed at 25° C. for 1 hour to immobilize the capture antibody. After the reaction solution was removed, 300 μL of a blocking agent (Blocking One) was added, and a reaction was allowed to proceed at 4 ° C. overnight. After the blocking agent was removed, the plate was washed three times with 300 μL of washing solution 1 (TBS-0.05% Tween20-1 mM DTT). After washing, 250 μL of a sample obtained by diluting a sample obtained by adding Claudin-5 micelles to human pooled serum derived from healthy subjects (BioIVT) to 0.0078125 to 2 ng or a human serum sample derived from each disease patient (BioIVT, ProteoGenex) with reaction solution 1 (0.5% BSA-TBS-1 mM

DTT) to 10% was added, and a reaction was allowed to proceed at 25° C. for 1.5 hours under shaking. After the reaction solution was removed, the plate was washed three times with 300 pL of washing solution 1 and twice with 300 pL of washing solution 2 (TBS-1 mM DTT). Subsequently, 50 pL of reaction solution 1 prepared so as to contain 0.5 pg/mL of a detection antibody against Claudin-5 (EPR7583) was added, and a reaction was allowed to proceed at 25° C. for 1 hour under shaking. After the reaction solution was removed, the plate was washed five times with 200 pL of washing solution 2. Further, 50 μL of reaction solution 1 prepared so as to contain 50 ng/mL of a biotin-labeled secondary antibody (Biotin-SP-AffiniPure Donkey Anti-Rabbit IgG(H+L)) was added, and a reaction was allowed to proceed at 25° C. for 1 hour under shaking. After the reaction solution was removed, the plate was washed five times with 200 pL of washing solution 2. After washing, 50 μL of reaction solution 1 prepared so as to contain 50 ng/mL of HRP-labeled streptavidin (Pierce High Sensitivity Streptavidin-HRP) was added, and a reaction was allowed to proceed at 25° C. for 30 minutes under shaking. After the reaction solution was removed, the plate was washed five times with 200 μL of TBS. Finally, 100 μL of a substrate (TMB Substrate Kit (Thermo Fisher, 34021)) was added, and a reaction was allowed to proceed at 25° C. for 30 minutes. After the reaction was stopped by adding 100 μL of 10% sulfuric acid (Nacalai, 13089-25), the absorbance at a wavelength of 450 nm was measured using a TriStar LB 941 plate reader. Based on the absorbance obtained from the samples containing Claudin-5 micelles, a calibration curve was generated using a 4-parameter logistic model, and the amount of Claudin-5 in the serum derived from each disease patient was calculated.

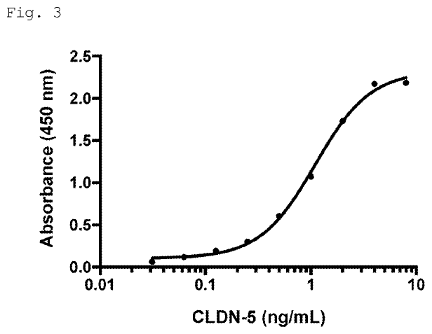

It was confirmed that Claudin-5 in human biological samples can also be detected by the immunoassay described above and quantified in the range of 0.0625 to 4 ng/mL (FIG. 3). The amounts of Claudin-5 in the sera of healthy subjects (Normal, n=4) and patients with Parkinson's disease (PD, n=2), multiple sclerosis (MS, n=2), Alzheimer's disease (AD, n=7), mild cognitive impairment (MCI, n=4), obsessive-compulsive disorder (OCD, n=1), and bipolar disorder (BD, n =2) as clinical specimens were measured. Interestingly, as shown in the results of Table 1 below, Claudin-5 was detected in four out of the four

MCI-derived serum specimens, and its calculated concentration was 0.886 to 1.520 ng/mL. In contrast, the concentration was below the detection limit when the clinical specimens derived from the healthy subjects or patients with other diseases were used.

TABLE 1

| | Conc. | | Conc. |
|---|---|---|---|
| Diagnosis | (ng/mL) | Diagnosis | (ng/mL) |
| Normal | ND | AD | ND |
| Normal | ND | AD | ND |
| Normal | ND | AD | ND |
| Normal | ND | AD | ND |
| PD | ND | AD | ND |
| PD | ND | AD | ND |
| MS | ND | AD | ND |
| MS | ND | MCI | 0.557 |
| OCD | ND | MCI | 1.412 |
| BD | ND | MCI | 0.886 |
| BD | ND | MCI | 1.520 |

As described above, the measurement system using the immunoassay established in this study can specifically detect Claudin-5, and Claudin-5 was detected in blood only in MCI before the onset of dementia. These results indicate that the presence or amount of Claudin-5 in body fluids, in particular, blood, is an indicator of MCI, which is a presymptomatic stage. It is interesting that Claudin-5 in blood was significantly correlated only with mild cognitive impairment, rather than with central nervous system diseases or mental disorders. Claudin-5 in body fluids, in particular, blood, is a biomarker specific for mild cognitive impairment and is believed to be an effective means that makes it possible to identify a subject who will be highly likely to have dementia in the future.

1. (canceled)

2. (canceled)

3. A method for testing for mild cognitive impairment in a subject using Claudin-5 in a body fluid collected from the subject as an indicator.

4. The method according to claim 3, wherein the testing comprises distinguishing mild cognitive impairment from a group of diseases consisting of Parkinson's disease, multiple sclerosis, Alzheimer's disease, obsessive-compulsive disorder, and bipolar disorder.

5. The method according to claim 3, comprising measuring the amount of Claudin-5 in the body fluid collected from the subject.

6. The method according to claim 5, wherein the amount of Claudin-5 in the body fluid is measured using an anti-Claudin-5 monoclonal antibody.

7. The method according to claim 3, comprising comparing the amount of Claudin-5 in the body fluid of the subject with the amount of Claudin-5 in a body fluid of a healthy subject.

8. The method according to claim 3, wherein the subject is a subject suspected of having cognitive impairment.

9. The method according to claim 3, further characterized by being combined with one or more diagnoses selected from the group consisting of a medical interview, a brain image analysis, a phosphorylated tau test, an amyloid β test, an electrophysiology study, a cerebrospinal fluid examination, a urinalysis, a blood test, a chest X-ray examination, a cerebral blood flow SPECT test, a brain glucose metabolism PET test, and a genetic test, with the proviso that a medical practice is excluded.

10. (canceled)


SEQUENCE LISTING (12 sequences)

SEQ ID NO: 1 - protein, 9 aa, Artificial
  HCDR1
GFSLNTYGV

SEQ ID NO: 2 - protein, 5 aa, artificial
  R9HCDR2
WWHGK

SEQ ID NO: 3 - protein, 8 aa, artificial
  R8HCDR3
ARAQLLDY

SEQ ID NO: 4 - protein, 17 aa, artificial
  R9LCDR1
KSSQSLLYSGDQKNYLA

SEQ ID NO: 5 - protein, 7 aa, artificial
  R9LCDR2
LTSTRES

SEQ ID NO: 6 - protein, 9 aa, artificial
  R9LCDR3
QQHYSYPLT

SEQ ID NO: 7 - protein, 7 aa, artificial
  2BHCDR1
GYTFTRF

SEQ ID NO: 8 - protein, 6 aa, artificial
  2BHCDR2
YPGSGD

SEQ ID NO: 9 - protein, 13 aa, artificial
  2BHCDR3
WGIYYGNPYAMDY

SEQ ID NO: 10 - protein, 16 aa, artificial
  2BLCDR1
RSSKSLLHSNGNTYLY

SEQ ID NO: 11 - protein, 7 aa, artificial
  2BLCDE2
RMSNLAS

SEQ ID NO: 12 - protein, 9 aa, artificial
  2BlCDR3
LQHLEYPFT